(19) United States
(12) Patent Application Publication
(10) Pub. No.: US 2023/0399294 A1
(43) Pub. Date: Dec. 14, 2023

(54) METHOD FOR SYNTHESIS OF MONOMER FOR SINGLE-ION BATTERY

(71) Applicants: BLUE SOLUTIONS CANADA INC., Boucherville (CA); CAPACITOR SCIENCES INCORPORATED, Menlo Park, CA (US)

(72) Inventors: Qiujie ZHAO, Menlo Park, CA (US); Siwei LIANG, Menlo Park, CA (US); Jin YANG, Menlo Park, CA (US); Sarah DEGRAS, BRossard (CA); Patrick LEBLANC

(21) Appl. No.: 18/207,057

(22) Filed: Jun. 7, 2023

Related U.S. Application Data
(60) Provisional application No. 63/350,119, filed on Jun. 8, 2022.

(51) Int. Cl. C07C 311/11

(52) U.S. Cl. CPC C07C 311/11; H01M 10/052

(57) ABSTRACT

The present technology relates to methods for the synthesis of a lithium single-ion monomer which comprises simultaneously reacting a sulfonyl chloride compound with i) a fluorinated sulfonamide compound and ii) a compound that is suitable to act as a quenching base and a lithium cation source. The simultaneous reaction of sulfonyl chloride with the fluorinated sulfonamide compound and the compound that is suitable to act as a quenching base and a lithium cation source yields the single-ion monomer.

### CROSS-REFERENCE TO RELATED APPLICATION

The present patent application claims the rights and benefits to U.S. Provisional Application No. 63/350,119, filed on Jun. 8, 2022, the content of which is incorporated herein by reference.

### TECHNICAL FIELD

The present technology generally relates to methods for the synthesis of lithium single-ion polymer electrolytes, and to methods for the synthesis of lithium single-ion monomers.

### BACKGROUND

A lithium battery using a lithium metal as a negative electrode has excellent energy density. However, with repeated cycles, such a battery can be subject to dendrites' growths on the surface of the lithium metal electrode when recharging the battery as the lithium ions are unevenly re-plated on the surface of the lithium metal electrode. To minimize the effect of the morphological evolution of the surface of the lithium metal anode including dendrites growth, a lithium metal battery typically uses a pressure system and a solid polymer electrolyte adapted to resist the pressure applied thereto as described in U.S. Pat. No. 6,007,935, incorporated herein by reference. Over numerous cycles, dendrites on the surface of the lithium metal anode, however, may still grow to penetrate the solid polymer electrolyte, and eventually cause ‘soft’ short circuits between the negative electrode and the positive electrode, resulting in decreasing or poor performance of the battery. Therefore, the growth of dendrites may still deteriorate the cycling characteristics of the battery and constitutes a major limitation with respect to the optimization of the performance of lithium batteries having a metallic lithium anode.

Various types of solid polymer electrolytes adapted for use with lithium metal electrodes have been developed since the late 1970s to overcome this issue but have been found to lack in conductivity and/or mechanical properties. Single-ion-conducting polymer electrolytes have, however, emerged as promising candidates, as the transference number of lithium cation approaches unity, and therefore prevents the formation of concentration gradients across the electrolyte, and dendrite formation as a result.

Existing methods of synthesis of lithium single-ion monomers comprise at least 3 steps (as represented in FIG. 1, and disclosed in ACS Appl. Mater. Interfaces 2016, 8, 10350-10359, incorporated herein by reference). A typical synthesis generally starts from a vinyl monomer (acrylate, methacrylate, styrene, etc.) bearing a sulfonate end group. The sulfonate is first converted into sulfonyl chloride using either thionyl chloride or oxalyl chloride. In the second step, trifluoromethanesulfonamide (NH₂SO₂CF₃) is attached to generate the (trifluoromethylsulfonyl)imide (TFSI) anion. In this step, as seen in FIG. 1, triethylamine (TEA) is used as a base to quench the byproduct HCl and acidic protons on the TFSI anions, resulting in a nitrogen-based organic cation, or more specifically, a triethylammonium cation, on the intermediate monomer. Finally, in the third step triethylammonium cations are substituted by lithium cations using LiH. The final product is then generally isolated by recrystallization (step not shown).

Other methods have also included exchanging the triethylammonium cations obtained by the methods disclosed above, with potassium cations, and then exchanging the potassium cation for a lithium cation in additional steps either before or after cross-linkage of the monomers (e.g., Polym. Chem., 2016, 7, 6901-6910; Journal of Polymer Science, 2020, 58, 2376-2388; and Electrochimica Acta, 2011, 57, 14-19, all incorporated herein by reference).

The methods disclosed above, however, suffer from several disadvantages. Existing methods generally use LiH in the ion-exchange step, which can be hazardous to handle in large quantities. In addition, the triethylammonium intermediate obtained by these methods is a dark brown oil which indicates the presence of a significant amount of impurities, which tend to stay with the recrystallized powder after the reaction with LiH and the recrystallization of the final product. Moreover, recrystallization of certain monomers bearing ionic groups can lead to self-polymerization which prevents copolymerization with different monomers and limits the uses and function of such monomers. Specifically, self-polymerization is considered to be an uncontrollable reaction wherein the final product is a mixture of very high molecular weight polymers and a small amount of monomers and oligomers. Therefore, control over the molecular weight of the final product in such settings is poor and affects the ionic conductivity of the final product.

Therefore, there is a need for alternative or improved methods of synthesis of lithium single-ion monomers which overcome or reduce at least some of the above-described problems.

### SUMMARY

From a broad aspect, the present technology relates to methods of synthesis of lithium single-ion monomers.

From one aspect there is provided a method for the synthesis of a lithium single-ion monomer which comprises simultaneously reacting a sulfonyl chloride compound with: i) a fluorinated sulfonamide compound; and ii) a compound that is suitable to act as a quenching base and a lithium cation source. The simultaneous reaction of sulfonyl chloride with the fluorinated sulfonamide compound and the compound that is suitable to act as a quenching base and a lithium cation source yields the single-ion monomer.

From another aspect, the present technology provides for a method for the synthesis of a lithium single-ion monomer, the method comprising simultaneously reacting a sulfonyl chloride compound with: i) a fluorinated sulfonamide compound; and ii) a compound that is suitable to act as a quenching base and a lithium cation source; wherein the simultaneous reaction of sulfonyl chloride with the fluorinated sulfonamide compound and the compound that is suitable to act as a quenching base and a lithium cation source yields the single-ion monomer; wherein the sulfonyl chloride compound is of formula:

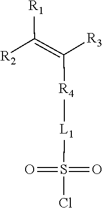

wherein: R₁ and R₂ are each independently H or F; R₃ is H, F, CN or CH₃; R₄ is an ester group, a phenyl group with L₁ substituted at ortho, para, or meta position, a fully or partially fluorinated phenyl group with L₁ substituted at ortho, para or meta position, an amide group, a carbonate group, or an ether group; and L₁ is a n-alkyl group (e.g., n=1-16), a fluorinated alkyl group (e.g., C₁-C₁₆), a branched alkyl group (e.g., C₃-C₁₆), an ethylene oxide linker (e.g., C₄ to C₁₆), a fluorinated ethylene oxide linker (e.g., C₄ to C₁₆), a cycloalkyl group (e.g., C₄ to C₇), a fluorinated cycloalkyl group (e.g., C₄ to C₇), or L₁ is absent. From another aspect, there is provided a method for the synthesis of a lithium single-ion monomer which comprises: 1) obtaining a sulfonyl chloride compound from a sulfonate; 2) simultaneously reacting a sulfonyl chloride compound with: i) a fluorinated sulfonamide; and ii) a compound that is suitable to act as a quenching base and a lithium cation source to obtain unpurified lithium single-ion monomer; and 3) purifying the unpurified lithium single-ion monomer.

From another aspect, the methods of the present technology are performed at reduced cost and have high atom economy (i.e., less reactant waste) compared to existing methods by virtue of comprising one step and bypassing the synthesis of the triethylammonium intermediate.

From another aspect, the methods of the present technology are safer to carry out compared to existing methods as the bases used in synthesis are safer to handle in scale-up production.

From another aspect the methods of the present technology result in a final product which is substantially free of impurities.

From another aspect the methods of the present technology overcome the self-polymerization of a lithium single-ion monomers.

### BRIEF DESCRIPTION OF THE DRAWINGS

Reference will now be made to the accompanying drawings.

FIG. 1 illustrates a typical 3-step synthesis of a lithium single-ion monomer according to Procarreli et al., ACS Appl. Mater. Interfaces 2016, 8, 10350-10359 (incorporated herein by reference).

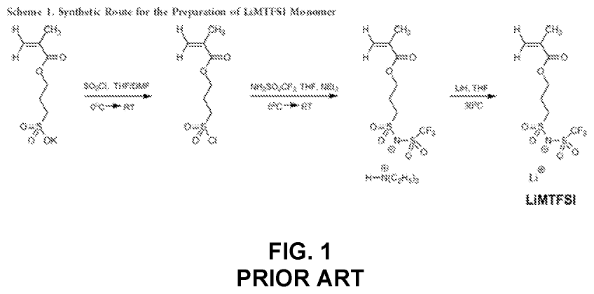

### DETAILED DESCRIPTION

### Definition

The use of “including”, “comprising”, or “having”, “containing”, “involving” and variations thereof herein, is meant to encompass the items listed thereafter as well as, optionally, additional items.

It must be noted that, as used in this specification and the appended claims, the singular form “a”, “an” and “the” include plural referents unless the context clearly dictates otherwise.

As used herein, the term “and/or” is to be taken as specific disclosure of each of the two specified features or components with or without the other. For example, “A and/or B” is to be taken as specific disclosure of each of (i) A, (ii) B and (iii) A and B, just as if each is set out individually herein.

As used herein, the term “about” in the context of a given value or range refers to a value or range that is within 20%, preferably within 10%, and more preferably within 5% of the given value or range.

As used herein the term “substantially” means to a great or significant extent.

As used herein, the expression “electron withdrawing group” refers to an atom or group that draws electron density from neighboring atoms towards itself, usually by resonance or inductive effects.

The present disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting.

Broadly, the present technology provides for methods of synthesis of lithium single-ion monomers which are simpler, safer, less costly and more efficient than existing methods of synthesis of lithium single-ion monomers.

In certain embodiments, the methods of the present technology comprise simultaneously reacting a sulfonyl chloride compound with i) a fluorinated sulfonamide compound and ii) a compound that is suitable to act as a quenching base and as a lithium cation source.

In some embodiments, the sulfonyl chloride compound has the following formula:

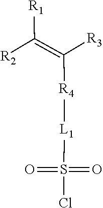

wherein: R₁ and R₂ are each independently H or F; R₃ is H, F, CN or CH₃; R₄ is an ester group, a phenyl group with L₁ substituted at ortho, para, or meta position, a fully or partially fluorinated phenyl group with L₁ substituted at ortho, para or meta position, an amide group, a carbonate group, or an ether group; and L₁ is a n-alkyl group (e.g., n=1-16), a fluorinated alkyl group (e.g., C₁-C₁₆), a branched alkyl group (e.g., C₃-C₁₆), an ethylene oxide linker (e.g., C₄ to C₁₆), a fluorinated ethylene oxide linker (e.g., C₄ to C₁₆), a cycloalkyl group (e.g., C₄ to C₇), a fluorinated cycloalkyl group (e.g., C₄ to C₇), or L₁ is absent.

In some instances, the ester group is —(C═O)—O— or —O—(C═O)—). In some instances, the amide group is —(C═O)—NH—. In some instances, the carbonate group is (—O—(C═O)—O—). In some instances, the ether group is (—O—).

In some embodiments, the fluorinated sulfonamide compound has the formula R_{A}—SO₂—NH₂, wherein R_{A} is selected from —F, —CF₃, —CF₂CF₃, —(CF₂)—CF₃, —C₆F₅, a branched C₃-C₄ fluoroalkyl group, such as —CF—(CF₃)₂, —CF(CF₃)—CF₂—CF₃, and CF₂—CF—(CF₃)₂, a linear perfluorethylether group, such as —(CF₂CF₂O)ₙ—CF₂CF₃, wherein n=1, 2 or 3, and an aryl compound substituted with at least one fluorine and at least one electron-withdrawing group.

In some embodiments the electron-withdrawing group is selected from —CN, —NO₂, —CF₃, and —SO₂CF₃.

In other embodiments, the aryl compound is —C₆F₄—CF₃, or —C₆F₄—SO₂CF₃, as disclosed in Huang et al. Chem. Mater. 2019, 31, 18, 7558 (incorporated herein by reference).

In the method of the present technology, the simultaneous reaction of the sulfonyl chloride with the fluorinated sulfonamide compound and the compound that is suitable to act as a quenching base and as a lithium cation source yields the lithium single-ion monomer in a single step.

In some embodiments, the methods of the present technology are a “one-pot synthesis”. As used herein, the expression “one-pot synthesis” refers to performing the methods of the present technology to successive chemical reactions in just one reactor. Performing the methods of the present technology into one reactor allows to avoid lengthy separation process and purification of the intermediate chemical compounds and can increase chemical yield.

In certain embodiments, as will be shown below, the fluorinated sulfonamide compound is trifluoromethanesulfonamide.

Thus far, TEA has been the base of choice in the synthesis of lithium single-ion monomers as weaker bases such as pyridine were found not to generate the target compound, and stronger bases such as NaH and EtONa gave side reactions and intensively colored and insoluble products (Shaplov et al., Polym. Chem., 2011, 2, 2609-2618, incorporated herein by reference). In addition, generation of the triethylamine counterion in the intermediate step has been the preferred route of synthesis as its removal can be easily monitored by ¹H NMR during recrystallization and purification of the final product.

The methods of the present technology, however, do not use TEA in the synthesis of the lithium single-ion monomers. Therefore, in certain embodiments, the methods of the present technology do not require the synthesis of a nitrogen-based organic cation. In other embodiments, the methods of the present technology do not require the synthesis of a stable nitrogen-based organic cation. In some embodiments, the nitrogen-based organic cation is a triethylammonium intermediate; therefore, the methods of the present technology do not require the synthesis of a triethylammonium intermediate. In other embodiments, the nitrogen-based organic cation is a stable triethylammonium intermediate; therefore, the methods of the present technology do not require the synthesis of a stable triethylammonium intermediate. In yet other embodiments, the nitrogen-based organic cation may be an alkylammonium such as R₅R₆R₇R₈N⁺, wherein R₅, R₆, R₇, and R₈ are independently selected from H, C₁-C₆ linear alkyl group, and C₃-C₆ branched alkyl groups. R₅, R₆, R₇, and R₈ can be either the same or different. In further embodiments, the nitrogen-based organic cation may be a substituted or unsubstituted imidazolium, pyridinium, piperidinium, and benzimizolium, wherein the substituents may be independently selected from H, C₁-C₆ linear alkyl group, and C₃-C₆ branched alkyl groups; and can be the same or different. In yet further embodiments, the nitrogen-based organic cation may be 1,2,3-triazolium or 1,2,4-triazolium. Therefore, in certain embodiments, the methods of the present technology do not require the synthesis of any one or more of an alkylammonium, imidazolium, pyridinium, piperidinium, benzimizolium, 1,2,3-triazolium, and 1,2,4-triazolium. Advantageously, by virtue of bypassing the synthesis of the nitrogen-based organic cation, the methods of the present technology offer a cheaper method of synthesis of lithium single-ion monomers and produce less reactant waste compared to existing methods.

In certain embodiments, the compound that is suitable to act as a quenching base and a lithium cation source is a lithium-containing basic compound. In some embodiments, the lithium-containing basic compound may be selected from Li₂CO₃, LiOH, Li₂SO₃, Li₃PO₄, lithium acetate, and lithium formate, and combinations thereof. In one embodiment, the lithium-containing basic compound is LiOH. Traditionally, use of a base such as LiOH, especially in the synthesis of acrylate and methacrylate derived monomers, would have been avoided due to the potential hydrolysis of the ester in the acrylate and methacrylate by LiOH. However, without being bound by theory, the investigators of the present technology have found that under anhydrous reaction conditions and the short reaction time required by the methods of the present technology, LiOH does not significantly hydrolyze the acrylates. This is believed to be partly due to the relatively neutral pH in the reaction, in which LiOH is neutralized by the HCl byproduct before it can start to hydrolyze said acrylates. In addition, use of LiOH in the methods of the present technology provides for a safer method of synthesis of lithium single-ion monomers compared to existing ones, as LiOH is safe to handle in large quantities and is particularly suitable for scale-up production of lithium single-ion monomers.

In certain embodiments, the fluorinated sulfonamide compound and the compound that is suitable to act as a quenching base and a lithium cation source are dissolved in an anhydrous solvent and mixed together prior to the addition of the sulfonyl chloride compound. In some embodiments, the anhydrous solvent may be selected from anhydrous methyl cyanide (MeCN), tetrahydrofuran (THF), acetone, dimethylformamide (DMF), and dimethyl sulfoxide (DMSO). Aqueous solvents such as water, alcohol and amine-containing solvents are not considered suitable for the methods of the present technology as water and hydroxy groups can react with the sulfonyl chloride to produce sulfonic acid, which is not the desired product. Therefore, in certain embodiments, the fluorinated sulfonamide compound and the compound that is suitable to act as a quenching base and a lithium cation source are not dissolved in an aqueous solvent.

In certain embodiments, the fluorinated sulfonamide compound and the compound that is suitable to act as a quenching base and a lithium cation source are mixed together at a temperature of from between about 15° C. and about 30° C. In other embodiments, the fluorinated sulfonamide compound and the compound that is suitable to act as a quenching base and a lithium cation source are mixed together at a temperature of about 15° C., about 20° C., about 25° C. (i.e., room temperature (RT)), or about 30° C. In some embodiments, the fluorinated sulfonamide compound and the compound that is suitable to act as a quenching base and a lithium cation source are mixed together at a temperature of about 25° C. (RT).

In other embodiments, the fluorinated sulfonamide compound and the compound that is suitable to act as a quenching base and a lithium cation source are mixed together from about 30 minutes to about 2 hours. In other embodiments, the fluorinated sulfonamide compound and the compound that is suitable to act as a quenching base and a lithium cation source are mixed together for about 30 minutes, for about 45 minutes, for about 1 hour, for about 1.5 hours, or for about 2 hours. In some embodiments, the fluorinated sulfonamide compound and the compound that is suitable to act as a quenching base and a lithium cation source are mixed together for about 1 hour.

In certain embodiments, the mixture of the fluorinated sulfonamide compound and the compound that is suitable to act as a quenching base and a lithium cation source is cooled to a temperature of from about −5° C. and about +4° C. prior to the addition of the sulfonyl chloride compound.

In some embodiments, the mixture of the fluorinated sulfonamide compound and the compound that is suitable to act as a quenching base and a lithium cation source is cooled to a temperature of about 0° C. The temperature of the mixture can be cooled to from about −5° C. to about +4° C., or to about 0° C., using known techniques such as an ice-water bath or the like.

In some embodiments, the sulfonyl chloride compound may also be dissolved in an anhydrous solvent prior to its addition to the mixture of the fluorinated sulfonamide compound and the compound that is suitable to act as a quenching base and a lithium cation source. In certain embodiments, the anhydrous solvent is the same as the anhydrous solvent used to dissolve the fluorinated sulfonamide compound and the compound that is suitable to act as a quenching base and a lithium cation source. In other embodiments, the anhydrous solvent in which the sulfonyl chloride is dissolved is different than the anhydrous solvent used to dissolve the fluorinated sulfonamide compound and the compound that is suitable to act as a quenching base and a lithium cation source. In such embodiments, the two different solvents are miscible in one another. In some embodiments, the sulfonyl chloride compound is dissolved in an anhydrous solvent selected from anhydrous MeCN, THF, acetone, DMF, and DMSO. For the same reasons provided above, the sulfonyl chloride compound is not dissolved in an aqueous solvent.

In certain embodiments, the mixture of the sulfonyl chloride and the anhydrous solvent is added to the mixture of the fluorinated sulfonamide compound and the compound that is suitable to act as a quenching base and a lithium cation source slowly and/or in a dropwise fashion. This prevents temperature jumps and solvent evaporation, and allows for the HCl by-product to be quenched efficiently by the quenching base.

The methods of the present technology, however, are not limited to a particular order in which the reagents are added. Therefore, in other embodiments, the mixture of the fluorinated sulfonamide compound and the compound that is suitable to act as a quenching base and a lithium cation source dissolved in the anhydrous solvent may be added to the sulfonyl chloride compound previously dissolved in an anhydrous solvent.

In other embodiments, the reaction of the sulfonyl chloride with the fluorinated sulfonamide and the compound that is suitable to act as a quenching base and a lithium cation source is carried out at a starting temperature of from about −5° C. and brought up to about +30° C. In some embodiments, said reaction is carried out at a starting temperature of from about 0° C. and brought up to about 25° C. In other embodiments, said reaction is carried out for about 30 minutes to about 5 hours. In further embodiments, said reaction is carried out for about 30 minutes to about 1 hour, about 30 minutes to about 1.5 hours, about 1.5 hours to about 3.5 hours, about 2 hours to about 3 hours, or about 2 hours to about 4 hours. In some embodiments, said reaction is carried out for about 2.5 hours. In another embodiment, said reaction is carried out for about 3 hours. In yet another embodiment, said reaction is carried out for about 30 minutes. In further embodiments, said reaction is carried out at a starting temperature of about 0° C. and brought up to about 25° C. for about 2.5 hours. In other embodiments, said reaction is carried out at a starting temperature of about 0° C. and brought up to about 25° C. for about 3 hours. In yet other embodiments, said reaction is carried out at a starting temperature of about 0° C. and brought up to about 25° C. for about 30 minutes.

In some embodiments, the sulfonyl chloride compound has formula:

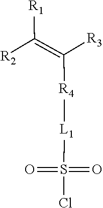

wherein R₁ and R₂ are each independently H or F; R₃ is H, F, CN or CH₃; R₄ is an ester group, a phenyl group with L₁ substituted at ortho, para, or meta position, a fully or partially fluorinated phenyl group with L₁ substituted at ortho, para, or meta position, an amide group, a carbonate group, or an ether group; L₁ is a n-alkyl group (e.g., n=1-16), a fluorinated alkyl group (e.g., C₁-C₁₆), a branched alkyl group (e.g., C₃-C₁₆), an ethylene oxide linker (e.g., C₄ to C₁₆), a fluorinated ethylene oxide linker (e.g., C₄ to C₁₆), a cycloalkyl group (e.g., C₄ to C₇), a fluorinated cycloalkyl group (e.g., C₄ to C₇), or L₁ is absent.

In some instances, the ester group is —(C═O)—O— or —O—(C═O)—). In some instances, the amide group is —(C═O)—NH—. In some instances, the carbonate group is (—O—(C═O)—O—). In some instances, the ether group is (—O—).

In certain embodiments, the sulfonyl chloride compound is converted from a sulfonate.

In certain embodiments, the sulfonate comprises a vinyl monomer such as acrylate, methacrylate, styrene or vinyl acetate. In other embodiments, the sulfonate has the formula:

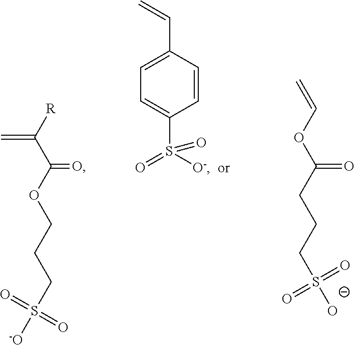

wherein R is H or CH₃.

In certain embodiments the cation associated with the sulfonate may be a monovalent cation selected from H⁺, K⁺, Na⁺, Li⁺, Rb⁺, and Cs⁺.

In certain embodiments, the sulfonyl chloride has the formula:

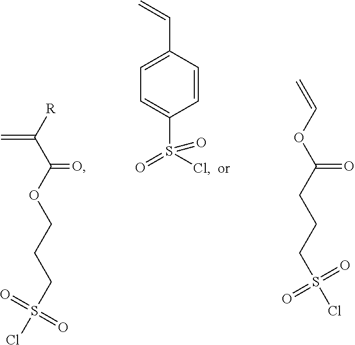

wherein R is H or CH₃.

In some embodiments, the conversion of the sulfonate to sulfonyl chloride is performed using one or more of thionyl chloride and oxalyl chloride. In certain embodiments, said conversion is carried out at a starting temperature of about −5° C. and brought up to about +30° C. In some embodiments, said conversion is carried out at a starting temperature of about 0° C. and brought up to about 25° C. In other embodiments, said conversion is carried out for about 12 hour to about 36 hours. In further embodiments, said conversion is carried out for about 14 hours to about 18 hours, about 20 hours to about 26 hours. In some embodiments, said conversion is carried out for about 16 hours. In other embodiments, said conversion is carried out for about 24 hours. In other embodiments, said conversion is carried out at a starting temperature of about 0° C. and brought up to about 25° C. for about 16 hours. In further embodiments, said conversion is carried out at a starting temperature of about 0° C. and brought up to about 25° C. for about 24 hours.

In certain embodiments, the methods of the present technology further comprise a step of purifying the lithium single-ion monomer. In certain implementations of these embodiments, the step of purifying the lithium single-ion monomer includes purifying by silica gel flash chromatography or by recrystallization.

In some embodiments, the lithium single-ion monomer is purified by silica gel flash chromatography. In certain implementations of these embodiments, the step of purifying further includes leaving residual solvent with the monomers to lower the concentration of the monomers in the final product. Advantageously, purification by silica gel flash chromatography prevents self-polymerization of the single-ion monomers, and self-polymerization is further alleviated by leaving residual solvent in the final product. Moreover, purification by silica gel flash chromatography allows for the colored impurities to be removed. As a result, the final lithium single-ion monomers obtained by the methods of the present technology is an almost clear viscous oil (with residual solvent).

In other embodiments, inhibitors preventing self-polymerization may be used in the step of purifying the lithium single-ion monomer. In certain embodiments, the inhibitors may be added to the column elution fractions that contain the pure product during silica gel flash chromatography. In such embodiments, the elution solvent may be removed by rotavap, leaving monomers well mixed with the inhibitors.

Inhibitors suitable for the methods of the present technology include 4-methoxyphenol (also referred to as MEHQ) and butylated hydroxytoluene (BHT). In some embodiments, the inhibitors may be used at ppm levels including from about 100 ppm to about 500 ppm. In some embodiments, about 3 mg to about 5 mg of MEHQ may be added to about 20 g of product to prevent self-polymerization.

In other embodiments, the step of purifying the lithium single-ion monomer comprises removing the LiCl byproduct before purifying by silica gel flash chromatography. In certain implementations of these embodiments, the step of removing the LiCl includes filtering the reaction product before running the silica gel flash chromatography.

In other embodiments, the methods of the present technology further comprise polymerizing the lithium single-ion monomer to obtain a lithium single-ion polymer. In certain embodiments polymerization may be performed by controlled polymerization (ATRP (“Atom Transfer Radical Polymerization”), RAFT (“Reversible Addition Fragmentation Chain Transfer”), anionic polymerization, cationic polymerization, free radical polymerization, or NMP (“Nitroxide-Mediated Radical Polymerization”)). In the methods of the present technology, polymerization of the final lithium single-ion monomer resulted in a white powder with no colored impurities.

In certain embodiments, the methods of the present technology comprise the following steps:

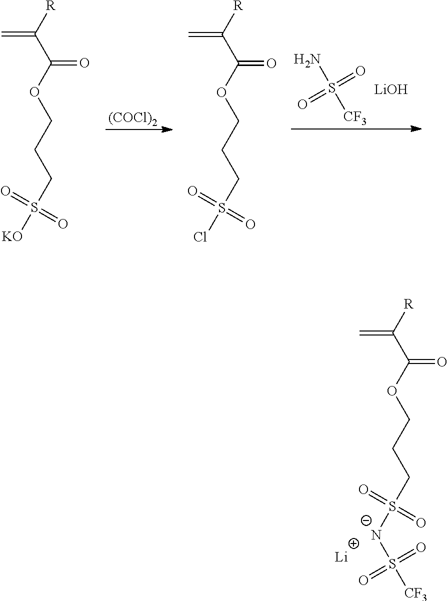

wherein R is H or CH₃.

In such embodiments, the lithium single-ion monomer obtained has formula:

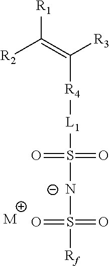

wherein R₁ and R₂ are independently H or F; R₃ is H, F, CN or CH₃; R₄ is an ester group, a phenyl group with L₁ substituted at ortho, para, or meta position, a fully or partially fluorinated phenyl group with L₁ substituted at ortho, para, or meta position, an amide group, a carbonate group, or an ether group; L₁ is a n-alkyl group (e.g., n=1-16), a fluorinated alkyl group (e.g., C₁-C₁₆), a branched alkyl group (e.g., C₃-C₁₆), an ethylene oxide linker (e.g., C₄ to C₁₆), a fluorinated ethylene oxide linker (e.g., C₄ to C₁₆), a cycloalkyl group (e.g., C₄ to C₇), a fluorinated cycloalkyl group (e.g., C₄ to C₇), or L₁ is absent; R_{f} is selected from F, CF₃, CF₂CF₃, (CF₂)ₙCF₃, C₆F₅, a branched C₃-C₄ fluoroalkyl group, —(CF₂CF₂O)ₙ—CF₂CF₃ wherein n=1, 2 or 3, and an aryl substituted with at least one fluorine and at least one electron-withdrawing group; and the electron withdrawing group is selected from —CN, —NO₂, —CF₃, and —SO₂CF₃; and M is monovalent cation H⁺, Li⁺, Na⁺, K⁺, Rb⁺ or Cs⁺.

In some instances, the ester group is —(C═O)—O— or —O—(C═O)—). In some instances, the amide group is —(C═O)—NH—. In some instances, the carbonate group is (—O—(C═O)—O—). In some instances, the ether group is (—O—).

In other embodiments, the methods of the present technology comprise 1) obtaining a sulfonyl chloride compound from a sulfonate; 2) simultaneously reacting a sulfonyl chloride compound with: i) a fluorinated sulfonamide compound; and ii) a compound that is suitable to act as a quenching base and a lithium cation source to obtain unpurified lithium single-ion monomer; and 3) purifying the unpurified lithium single-ion monomer, as described above. In certain embodiments, the fluorinated sulfonamide compound has the formula R_{A}—SO₂—NH₂, wherein R_{A} is selected from —F, —CF₃, —CF₂CF₃, —(CF₂)ₙCF₃, —C₆F₅, a branched C₃-C₄ fluoroalkyl group, such as —CF—(CF₃)₂, —CF(CF₃)—CF₂—CF₃, and CF₂—CF—(CF₃)₂, a linear perfluorethylether group, such as —(CF₂CF₂O)ₙ—CF₂CF₃, wherein n=1, 2 or 3, and an aryl compound substituted with at least one fluorine and at least one electron-withdrawing group. In some embodiments the electron-withdrawing group is selected from —CN, —NO₂, —CF₃, and —SO₂CF₃. In other embodiments, the aryl compound is —C₆F₄—CF₃, or —C₆F₄—SO₂CF₃.

In certain embodiments, the mass yield of the lithium single-ion monomer obtained by the methods of the present technology is between about 60% and about 99%, between about 70% and about 80%, between about 80% and 99%, about 75%, or about 95%.

### EXAMPLES

The examples below are given to illustrate the practice of various embodiments of the present disclosure. They are not intended to limit or define the entire scope of this disclosure.

It should be appreciated that the subject matters of this disclosure are not limited to the particular embodiments described and illustrated herein but includes all modifications and variations falling within the scope of the subject matters of this disclosure as defined in the appended claims.

### Example 1: Synthesis of lithium 1-[3-(acryloyloxy)-propylsulfonyl]-1-(trifluoromethylsulfonyl)imide (LiATFSI)

In an Ar-filled, magnet-stir bar equipped 500 mL round bottom flask, anhydrous DMF (2.8 g, 0.038 mol) was dissolved in 50 mL anhydrous MeCN. The flask was cooled to 0° C. with an ice-water bath and oxalyl chloride (20.8 g, 0.164 mol) was added dropwise via syringe with stirring. 20 mL anhydrous MeCN was added after to dilute the solution and the ice bath was removed to allow the reaction to be stirred at room temperature for 1 hour. Once there was no bubbles forming, the reaction was cooled to 0° C. again. 3-sulfopropyl acrylate potassium salt (30 g, 0.129 mol) was suspended in 75 mL MeCN and added portion-wise via a funnel under an Ar stream and vigorous stirring. An additional 25 mL MeCN was used to wash off residual solid from the weighing container and added to the reaction flask. The mixture was stirred at room temperature for 16 hours. The reaction was then poured into 200 mL ice-cold deionized water in a separation funnel. The bottom water layer was collected and washed three times with 50 mL dichloromethane (DCM). The top organic layer was collected and combined with the 150 mL DCM solution. This organic solution was further washed three times with 50 mL deionized (DI) water and dried with anhydrous MgSO₄. Then it was filtered and the clear filtrate was reduced to a slightly yellow liquid via rotavap. 5 mg MEHQ inhibitor was added to the crude 3-(chlorosulfonyl) propyl acrylate (J501, as indicated in the synthetic route below) and stored at 4° C. for further use.

To synthesize lithium LiATFSI (J503), lithium hydroxide (1.3 g, 54 mmol) and trifluoromethanesulfonylamide (4.0 g, 27 mmol) were dissolved in 25 mL anhydrous MeCN in an Ar-filled flask equipped with a magnetic stir bar. The mixture was stirred at room temperature for 1 hour before cooling to 0° C. using an ice-water bath. Then 3-(chlorosulfonyl) propyl acrylate (5.7 g, 27 mmol) was dissolved in 12 mL anhydrous MeCN and the solution was added dropwise to the reaction flask. The mixture was then stirred from 0° C. to room temperature for 2.5 hours. After the reaction, the white precipitates (LiCl salt) was filtered off and the filtrate was reduced and purified by a silica gel column. LiATFSI was eluted from pure DCM to DCM+20 vol % THF. The product fractions were combined and 4 mg MEHQ inhibitor was added. The solvent was reduced to about 50 wt % compared to the LiATFSI and the viscous solution was stored at 4° C. for further use. The residual solvent content and monomer purity were quantified by ¹H NMR integration. The final product contained 6.6 g LiATFSI and 3.4 g residual solvents (DCM+THF). The reaction yield was 74%.

The synthetic route for the preparation of LiATFSI according to this embodiment is represented below.

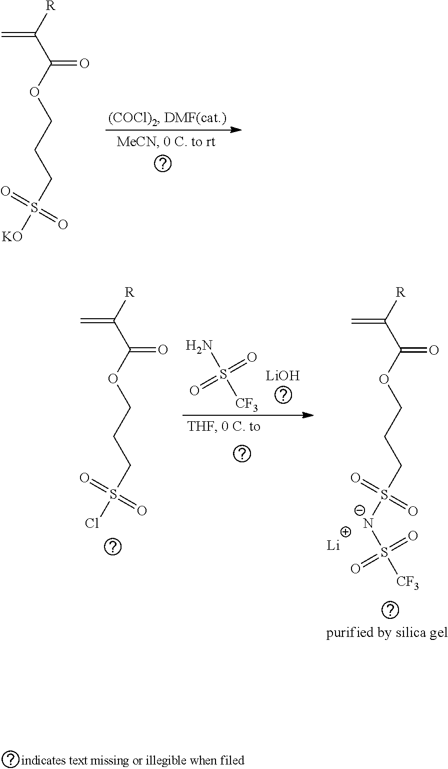

### Example 2: Synthesis of lithium 1-[3-(methacryloyloxy) propylsulfonyl]-1-(trifluoromethylsulfonyl)imide (LiMTFSI)

To a 250 mL three-neck flask equipped with gas inlet adapters with continuously flowing Ar stream, were added 70 mL anhydrous MeCN and 18.57 g (0.146 mol) oxalyl chloride. The mixture was cooled down with an ice bath and 2 mL DMF was added dropwise via syringe while stirring. After addition, the ice bath was removed and the mixture was allowed to stir at room temperature for 1 hour. Afterwards, the reaction mixture was cooled down with ice bath again and 3-sulfopropyl methacrylate potassium salt (22.7 g, 0.09 mol) was added slowly to the above mixture. The reaction mixture was allowed to slowly warm up to room temperature and stir for 24 hours. Then the reaction mixture was filtrated to remove KCl and the filtrate was condensed by rotavap. The residue liquid was diluted by 50 mL DCM and the DCM solution was washed by 50 mL DI water. The aqueous phase was washed three times with 30 mL of DCM. The organic phases were combined and dried over anhydrous MgSO₄. After filtration, the filtrate was condensed and purified by flash column chromatography with DCM as eluent solvent to give 3-(chlorosulfonyl) propyl methacrylate (J509, as indicated in the synthetic route below) as a light yellow liquid (14.5 g, 71%). To synthesize LiMTFSI (J505, as indicated in the synthetic route below) 2.95 g lithium hydroxide (0.12 mol) and 8.9 g trifluoromethanesulfonylamide (59 mmol) were added into a 250 mL three-neck flask equipped with an additional funnel and gas inlet adapters under Ar atmosphere, followed by 40 mL anhydrous THF. The mixture was cooled down to 0° C. by an ice bath. Then a mixture of J509 (13.54 g, 59 mmol) and 40 mL anhydrous THE was added Use Gap Code dropwise to the reaction solution. Then the reaction mixture was slowly warmed up to room temperature with stirring for 3 hours. After the reaction, the solution was condensed via vacuum and then diluted by DCM. The white solid was removed by filtration and the solvent was evaporated by rotavap to give J505 as a light yellow liquid which contained 19.6 g of LiMTFSI. The reaction yield was 95%.

The synthetic route for the preparation of LiMTFSI according to this embodiment is represented below.

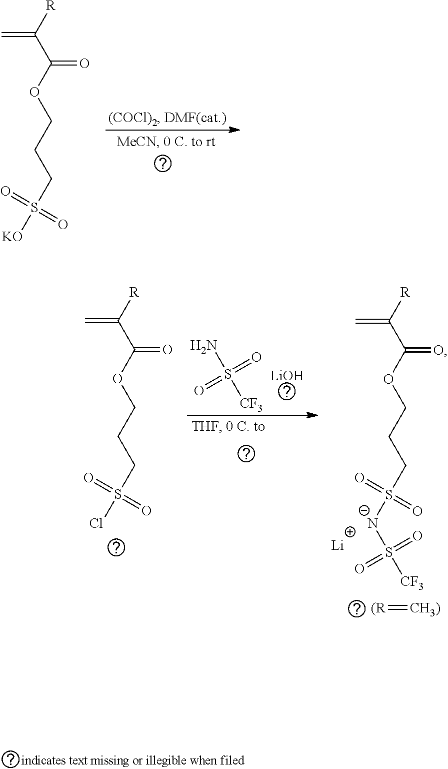

Variations and modifications will occur to those of skill in the art after reviewing this disclosure. The disclosed features may be implemented, in any combination and subcombinations (including multiple dependent combinations and subcombinations), with one or more other features described herein. The various features described or illustrated above, including any components thereof, may be combined or integrated in other systems. Moreover, certain features may be omitted or not implemented. Examples of changes, substitutions, and alterations are ascertainable by one skilled in the art and could be made without departing from the scope of the information disclosed herein.

It should be appreciated that the present technology is not limited to the particular embodiments described and illustrated herein but includes all modifications and variations falling within the scope of the present technology as defined in the appended claims.

All references cited in this specification, and their references, are incorporated by reference herein in their entirety where appropriate for teachings of additional or alternative details, features, and/or technical background.

1. A method for the synthesis of a lithium single-ion monomer, the method comprising simultaneously reacting a sulfonyl chloride compound with:
i) a fluorinated sulfonamide compound; and
ii) a compound that is suitable to act as a quenching base and a lithium cation source;
wherein the simultaneous reaction of sulfonyl chloride with the fluorinated sulfonamide compound and the compound that is suitable to act as a quenching base and a lithium cation source yields the single-ion monomer; wherein the sulfonyl chloride compound is of formula: 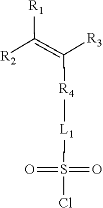
wherein
R₁ and R₂ are each independently H or F;
R₃ is H, F, CN or CH₃;
R₄ is an ester group, a phenyl group with L₁ substituted at ortho, para, or meta position, a fully or partially fluorinated phenyl group with L₁ substituted at ortho, para or meta position, an amide group, a carbonate group, or an ether group; and
L₁ is a n-alkyl group, a fluorinated alkyl group, a branched alkyl group, an ethylene oxide linker, a fluorinated ethylene oxide linker, a cycloalkyl group, a fluorinated cycloalkyl group, or L₁ is absent.

2. The method of claim 1, wherein the fluorinated sulfonamide compound has a formula R_{A}—SO₂—NH₂, wherein R_{A} is selected from F, CF₃, CF₂CF₃, (CF₂)ₙCF₃, C₆F₅, a branched C₃-C₄ fluoroalkyl group, —(CF₂CF₂O)ₙ—CF₂CF₃ wherein n=1, 2 or 3, and an aryl substituted with at least one fluorine and at least one electron-withdrawing group.

3. The method of claim 2, wherein the electron withdrawing group is selected from —CN, —NO₂, —CF₃, and —SO₂CF₃.

4. The method of claim 1, wherein the method does not require the synthesis of a nitrogen-based organic cation.

5. The method of claim 1, wherein the method does not require the synthesis of a stable nitrogen-based organic cation.

6. The method of claim 1, wherein the compound that is suitable to act as a quenching base and a lithium cation source is a lithium-containing basic compound.

7. The method of claim 6, wherein the lithium-containing basic compound is selected from Li₂CO₃, LiOH, Li₂SO₃, Li₃PO₄, lithium acetate, and lithium formate, and combinations thereof.

8. The method of claim 1, wherein the sulfonate compound is of formula: 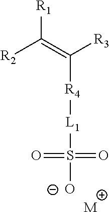
wherein
R₁ and R₂ are each independently H or F;
R₃ is H, F, CN or CH₃;
R₄ is an ester group, a phenyl group with L₁ substituted at ortho, para, or meta position, a fully or partially fluorinated phenyl group with L₁ substituted at ortho, para, or meta position, an amide group, a carbonate group, or an ether group;
L₁ is a n-alkyl group, a fluorinated alkyl group, a branched alkyl group, an ethylene oxide linker, a fluorinated ethylene oxide linker, a cycloalkyl group, a fluorinated cycloalkyl group, or L₁ is absent; and
M is monovalent cation H⁺, Li⁺, Na⁺, K⁺, Rb⁺ or Cs⁺.

9. The method of claim 8, wherein the conversion from sulfonate to sulfonyl chloride is performed using one or more of thionyl chloride and oxalyl chloride.

10. The method of claim 1, wherein the sulfonyl chloride compound has the formula: 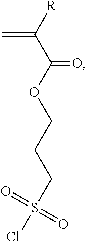
wherein R is H or CH₃,

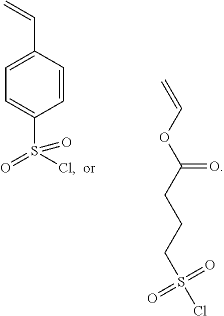

11. The method of claim 8, wherein the sulfonate has the formula: 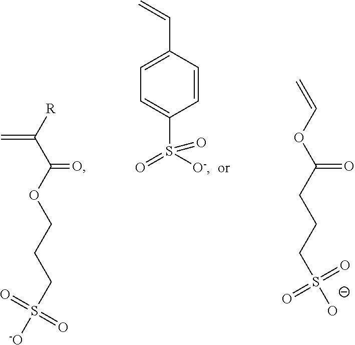
wherein R is H or CH₃.

12. The method of claim 1, wherein the lithium single-ion monomer has formula: 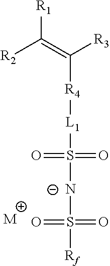
wherein
R₁ and R₂ are independently H or F;
R₃ is H, F, CN or CH₃;
R₄ is an ester group, a phenyl group with L₁ substituted at ortho, para, or meta position, a fully or partially fluorinated phenyl group with L₁ substituted at ortho, para, or meta position, an amide group, a carbonate group, or an ether group;
L₁ can be a n-alkyl group, a fluorinated alkyl group, a branched alkyl group, an ethylene oxide linker, a fluorinated ethylene oxide linker, a cycloalkyl group, a fluorinated cycloalkyl group, or L₁ is absent;
R_{f} is selected from F, CF₃, CF₂CF₃, (CF₂)ₙCF₃, C₆F₅, a branched C₃-C₄ fluoroalkyl group, —(CF₂CF₂O)ₙ—CF₂CF₃ wherein n=1, 2 or 3, and an aryl substituted with at least one fluorine and at least one electron-withdrawing group; and the electron withdrawing group is selected from —CN, —NO₂, —CF₃, and —SO₂CF₃; and
M is monovalent cation H, Li⁺, Na⁺, K⁺, Rb⁺ or Cs⁺.

13. The method of claim 1, further comprising a step of purifying the lithium single-ion monomer.

14. The method of claim 13, wherein the step of purifying includes removing the LiCl byproduct and running a silica gel flash chromatography.

15. The method of claim 13, further comprising polymerizing the lithium single-ion monomer.

16. The method of claim 1, wherein the method comprises the following steps: 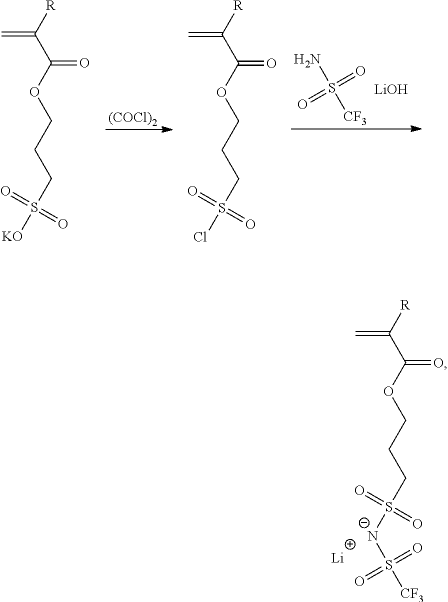
wherein R is H or CH₃.

17. A method for the synthesis of a lithium single-ion monomer, the method comprising:
1) obtaining a sulfonyl chloride compound from a sulfonate;
2) simultaneously reacting the sulfonyl chloride compound with:
i) a fluorinated sulfonamide compound and
ii) a compound that is suitable to act as a quenching base and a lithium cation source to obtain unpurified lithium single-ion monomer; and
3) purifying the unpurified lithium single-ion monomer.

18. The method of claim 17, wherein the fluorinated sulfonamide compound has the formula R_{A}—SO₂—NH₂, wherein R_{A} is selected from F, CF₃, CF₂CF₃, (CF₂)ₙCF₃, C₆F₅, a branched C₃-C₄ fluoroalkyl group, —(CF₂CF₂O)ₙ—CF₂CF₃ wherein n=1, 2 or 3, and an aryl substituted with at least one fluorine and at least one electron-withdrawing group.

19. The method of claim 18, wherein the electron withdrawing group is selected from —CN, —NO₂, —CF₃, and —SO₂CF₃.
